(19) United States
(12) Patent Application Publication
(10) Pub. No.: US 2010/0008908 A1
(43) Pub. Date: Jan. 14, 2010

(54) Treatment of Heart Failure

(75) Inventors: Houman Ashrafian, Headington (GB); Hugh Watkins, Headington (GB)

Correspondence Address: ROTHWELL, FIGG, ERNST & MANBECK, P.C., 1425 K STREET, N.W., SUITE 800, WASHINGTON, DC, 20005, US

(73) Assignee: Isis Innovation Limited, Summertown (GB)

(21) Appl. No.: 12/417,247

(22) Filed: Apr. 2, 2009

(30) Foreign Application Priority Data
Apr. 3, 2008 (GB) 0806047.7

(51) Int. Cl. A61K 39/395; A61K 31/138; A61K 31/7088; A61P 9/04; C07C 211/26; C07H 21/02; C07K 16/00

(52) U.S. Cl. 424/133.1; 514/648; 514/44 R; 514/44 A; 424/141.1; 564/315; 536/23.1; 530/387.3; 530/388.1

(57) ABSTRACT

The treatment of heart failure by administering a therapeutically effective amount of an agent that inhibits hypoxia-inducible factor (HIF). Such agents include small molecule chemical agents and biological agents.

This application claims priority from GB patent application number 0806047.7, filed Apr. 3, 2008. This prior application is incorporated herein by reference.

### INTRODUCTION

The invention relates to the treatment of heart failure and disorders associated with heart failure. In particular the invention relates to the treatment of such conditions by inhibition of HIF-1.

### BACKGROUND

Hypoxia-inducible factor (HIF) is a transcription factor that has a central role in oxygen and energy homeostasis and plays a key role in response to hypoxia. It regulates the expression of a wide variety of genes including genes associated with angiogenesis, apoptosis and cellular metabolism. HIF is a heterodimer consisting of two subunits: the oxygen sensitive alpha subunit (HIF-α) and the constitutively expressed beta subunit (HIF-β). Both subunits belong to the family of helix-loop-helix transcription factors.

Three HIF-alpha homologues have been identified: HIF-1α, HIF-2α and HIF-3α. HIF-1α and HIF-2α show a high degree of similarity and are both able to bind HIF-β.

HIF regulation is complex. Although both α and β subunits are constitutively expressed, under normoxic conditions the ubiquitin proteasome acts to degrade the HIF-α protein (the HIF-α protein is modified by prolyl-hydroxylases, PHD1-3, on the oxygen-dependent degradation domain on residues 401 and 603; then becomes VHL bound, ubiquitinated and rapidly degraded by the 26S proteasome). However, under hypoxic conditions there is less degradation of HIF-α (specifically HIF1α and HIF2α) allowing the formation of α-β dimers and the activation of a variety of HIF-regulated genes which act to reduce oxygen utilization and increase oxygen delivery, such as heme oxygenase-1 (HO-1), nitric oxide synthases (NOSI/III), VEGF, glycolytic enzymes, glucose transporters and erythropoietin.

Because of the role of HIF in angiogenesis, many inhibitors of HIF-1α have been described for the treatment of cancer. An example of a publication describing such inhibitors is WO2008/004798.

The role of HIF-1 in the heart has also been described. HIF-1α is generally considered beneficial for the heart and there are reports that inhibition of HIF-1 causes cardiac dysfunction (Masanori et al. Nature (2007) 446, 444-448). Accordingly, there has been profound interest in augmenting HIF activity both in animal models and in humans to improve the response to cardiac ischaemia and infarction, and to retard the transition to left ventricular dysfunction and heart failure. However there are also reports that describe an association between heart failure and upregulated HIF-1α (Shyu et al. J Card Fail (2005) 11, 152-159; Rastogi et al. Circulation (2004) 110 (17S), 479; Liu et al. Mol Cell Biol, online publication 2008). Kakinuma et al (Circulation (2001) 103, 2387-2394) obtained data which suggested that HIF-1α may contribute to the changes seen in heart failure based on work in cells; nonetheless this work did not in any way suggest that HIF antagonism might be an effective therapy for heart failure.

Heart failure is a common condition affecting about 2% of the general population and up to 15% of the elderly population. It is a progressive disorder in which damage to the heart causes weakening of the cardiovascular system. It manifests by fluid congestion or inadequate blood flow to tissues. There are many different ways to categorize heart failure, including the side of the heart involved, (left heart failure versus right heart failure); whether the abnormality is due to contraction or relaxation of the heart (systolic dysfunction vs. diastolic dysfunction); whether the abnormality is due to low cardiac output or high systemic vascular resistance (low-output heart failure vs. high-output heart failure), and the degree of functional impairment conferred by the abnormality. Although successful drug therapies exist, the mortality rate associated with even well-treated heart failure is unacceptable. There is a shortage of new therapies in this area and a desperate need for novel approaches to treatment.

It is an object of the invention to provide a method for treating heart failure.

### SUMMARY OF THE INVENTION

In a first aspect the invention relates to a method of treating heart failure or disorders associated with heart failure in a patient suffering from such a condition which comprises administering to such a patient a therapeutically effective amount of an agent that inhibits hypoxia-inducible factor (HIF).

Preferably the agent inhibits HIF-1, for example HIF-1α. Preferably the agent inhibits the transcription of HIF, inhibits the translation of HIF, inhibits the subcellular localization of HIF, inhibits the activity of HIF, accelerates the degradation of HIF or decreases the stability of HIF.

According to one embodiment the agent is a small molecule inhibitor of HIF, for example taxol, rapamycin, 17-AAG (17-allylaminogeldanamycin), YC-1 (3-15′-hydroxymethyl-2′-furyl)-1-benzylindazole), adenosine, topotecan, imatinib mesylate, trastuzumab, NS398, celecoxib, ibuprofen, resteratiol, genistein, and berberin

According to other embodiments the agent may be a short hairpin RNA (shRNA) for a HIF gene, a microRNA for a HIF gene, an antisense oligonucleotide for a HIF gene, a small double stranded interference RNA (siRNA) directed against a HIF gene or a monoclonal antibody directed against a HIF protein.

The disease to be treated may be chronic heart failure. The disorder associated with heart failure may be diastolic heart failure or dilated, restrictive or hypertrophic cardiomyopathy.

In another aspect the invention relates to an agent that inhibits hypoxia-inducible factor (HIF) for the treatment of heart failure or disorders associated with heart failure.

In yet another aspect the invention relates to the use of an agent that inhibits hypoxia-inducible factor (HIF) for the manufacture of a medicament for the treatment of heart failure or disorders associated with heart failure.

### BRIEF DESCRIPTION OF DRAWINGS

The present invention will now be described with reference to the accompanying drawings:

FIG. 1 shows that PHD3 knockout causes at least moderate ventricular dysfunction in PHD3 knockout animals which were assessed for cardiac dysfunction to assess the effect of HIF upregulation. In particular, FIG. 1a shows the ejection fraction (EF) as judged by 2D echocardiography; FIG. 1b shows the assessment of LV systolic function by invasive hemodynamics; FIG. 1c shows the assessment of LV diastolic function by invasive hemodynamics, and FIG. 1d confirms impairment of LV dysfunction by a profound increase in LVEDP (p<0.0001).

### DETAILED DESCRIPTION OF THE INVENTION

The invention is based on the finding that HIF antagonism is an effective treatment for heart failure. While acute activation of HIF may be beneficial for the heart, it has now been found that chronic activation of HIF in heart failure is detrimental due to factors such as reduced mitochondrial function and increased apoptosis. Accordingly, in one embodiment the present invention provides a method of treating heart failure or disorders associated with heart failure in a patient suffering from such a condition which comprises administering to such a patient a therapeutically effective amount of an agent that inhibits HIF.

The method of the invention may be used to treat heart failure, including chronic heart failure of all aetiologies, and also disorders associated with heart failure, such as diastolic heart failure, and cardiomyopathies (dilated, restrictive and hypertrophic).

By “treating” we mean symptomatic improvement, which may consist of decreased breathlessness, improved exercise capacity, reduced fatigue and an improved psychological state; and/or disease modification with slowing, arresting or reversing of symptom and disease progression. This would ultimately result in a reduction in both dose and absolute necessity for other therapies (e.g. pharmacological, device, surgical, transplantation or otherwise), reduced hospitalisation and ultimately reduced mortality. By “an agent that inhibits HIF” we mean any agent that acts to inhibit the transcription of HIF, to inhibit the translation of HIF, to inhibit the subcellular localization of HIF, to inhibit the activity of HIF, to accelerate the degradation of HIF or to decrease the stability of HIF. Such agents will prevent the action of HIF as a transcription factor.

Preferably, the agent that inhibits HIF will inhibit HIF-1. More preferably, the agent that inhibits HIF will inhibit HIF-1 alpha.

Agents that inhibit HIF are well known. The agent may be a small molecule inhibitor of HIF. Small molecule inhibitors of HIF include natural and synthetic molecules such as taxol, rapamycin, 17-AAG (17-allylaminogeldanamycin), YC-1 (3-15′-hydroxymethyl-2′-furyl)-1-benzylindazole), adenosine, topotecan, imatinib mesylate, trastuzumab, NS398, celecoxib, ibuprofen, resteratiol, genistein, and berberin.

These and other such molecules are disclosed for example in WO2007/150011, WO2008/004798, WO2007/040565, WO2007/065010, WO2007/130037, WO2007/123777, Jones and Harris (Mol Cancer Ther (2006) 5, 21932202), Chau et al (Cancer Res (2005) 65, 4918-4928), Rapisarda et al (Cancer Res (2002) 62, 4316-4324).

It is also possible in accordance with the invention to administer a combination of two or more inhibitors. For example two inhibitors which inhibit HIF in different ways could be used. As an example a first inhibitor may alter the transcription of HIF, whilst a second inhibitor alters the degradation of HIF. Any combination of inhibitors may be used and is not restricted to the example above. Moreover, HIF inhibitors could be used freely in combination with other existing and future heart failure therapies.

It is possible to use the RNAi approach to directly or indirectly inhibit expression of a HIF gene, as would be apparent to the person skilled in the art. As an example of RNAi for inhibition of HIF-1α mRNA expression we refer to WO2007/076351.

An alternative approach uses microRNAs (miRNAs; miRs). miRs are small double stranded RNA molecules that are encoded in miRNA precursor genes. miRNA precursors mRNAs are transcribed; fold and are processed by the proteins Drosha and DICER to 20-25 base pair double-stranded RNA molecules. miRs negatively regulate expression of their target genes at the post-transcriptional level. miRNAs could be used in the method of the invention in accordance with the knowledge of the skilled person.

Yet another alternative approach uses small double stranded interference RNAs (siRNAs) directed against HIF. A similar effect can be achieved with or short hairpin RNAs (shRNAs).

A further approach is to use antisense technology to inhibit expression of a HIF gene, as would be apparent to the person skilled in the art.

Another approach is the use of monoclonal antibodies directed against the HIF proteins. Such monoclonal antibodies can be made by standard methods. Alternatively, DNA encoding monoclonal antibodies, antigen binding chains or domains can be cloned and expressed using standard methods of recombinant DNA technology. Recombinant antigen binding molecules can be manipulated to improve therapeutic properties such as specificity, affinity, half-life and lack of immunogenicity. Rodent (e.g. rat or mouse) or other non-human animal (e.g. horse) antibodies can be used according to the invention. However, for use in man it is preferred that the antibody has been engineered to limit the anti-globulin response. Examples of antibodies engineered in this way are chimeric antibodies (where the constant regions of a non-human antibody are replaced by human constant regions) and humanised antibodies where the antibody is engineered to appear human to the immune system of the recipient. Examples of humanised antibodies are CDR-grafted antibodies where as well as replacing the constant regions of a non-human antibody with a human constant region, the framework regions of the variable regions are also replaced by human variable regions.

The agent that inhibits HIF may be administered to the patient in a number of ways. For example use could be made of liposomes, nanoparticles, viral vectors, and the like. The best inhibitors would be orally administrable, although any other forms of administration could be used.

The agents described above should be administered to patients in therapeutically effective amounts. Such amounts and dosage regimes can be determined in the usual manner as known to the skilled person. The doses would either be administered adhering to standard treatment doses or will be adjusted to acceptable biomarker led levels.

For example, it is well recognised that HIF activation leads to the elaboration of a number of critical downstream mediators such as VEGF, BNIP3 and FoxO3a which may subserve the detrimental effects of HIF in heart failure. This is illustrated in the diagram below which summarises the role of HIF activation in heart failure (HF).

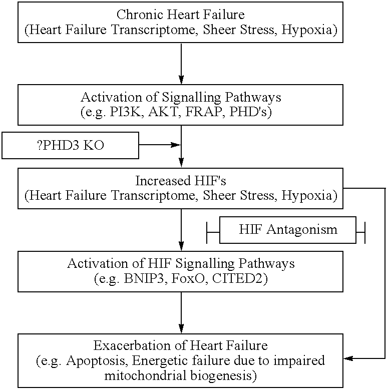

The state of chronic heart failure is associated with chronic activation of HIF. This may be in part due to the HF transcriptome, in response to mechanical stresses (Kim et al (Circulation Research. (2002) 90:e25.) and/or hypoxia. Increased HIF mRNA and/or persistence of HIF protein due to increased production or decreased degradation, will lead to increased transcription of a variety of downstream mediators such as BNIP3 or FoxO's (Bakker W et al. Molecular Cell. (2007) 28:941-53). These mediators, through energetic impairment, increased apoptosis or other pathways will exacerbate HF. Accordingly, inhibiting HIF or its direct downstream pathways will ameliorate HF.

The levels of these mediators may thus be used as a direct read-out for the correct dosing for HIF antagonists in heart failure. For example, systemic venous levels of VEGF are known to be increased in heart failure (B. S. Chin. Am J Cardiol 90 (2002), pp. 1258-1260). By adjusting the dosing of HIF antagonism to these systemic levels, patients may be directed to use optimal doses of these agents.

Alternatively, in the case that the systemic levels of these markers may not be adequately informative, the use of HIF antagonists may be better guided by direct measurements of venous effluent from the heart (e.g. coronary sinus effluent of HIF downstream targets) or from assessment of the heart muscle itself. While the immediate methodologies currently available to assess these parameters are based on invasive cardiac venous sampling or myocardial biopsy and may be clinically practicable, there is also significant potential for the application of bioimaging modalities to assess downstream targets with greater ease non-invasively.

For example, the use of HIF antagonists and their dosing may be guided by the use of PET tracers, antibody based assays, contrast/conventional echocardiographic studies or contrast/conventional MRI (including cardiac MRI techniques) to study the impact of cardiac HIF signalling. For example, it is known that enhanced HIF signalling augments local VEGF, VEGFR-1, VEGFR-2 in the heart (Post M J Am J Physiol Regul Integr Comp Physiol. (2006) 290:R494-500). Although this effect has not be studied in heart failure, there is considerable evidence that PET tracers such as VEGFR-2-specific tracers; 64Cu-DOTA-VEGFDEE and 64Cu-DOTA-VEGF12, may soon be translated into the clinic for imaging tumor angiogenesis and antiangiogenic treatment (Wang H Eur J Nucl Med Mol Imaging. (2007) 34:2001-10). This may guide HIF antagonism in heart failure. Moreover the use of reporter assays in the heart (a reporter gene for HIF pathway signalling introduced by gene therapy) coupled with PET or MR tracers are to be imminently available (Wu J C Radiology. (2007) 244:337-55). These may guide HIF therapy in heart failure and related disorders with HIF antagonists.

Other biomarkers that may also be useful might be related to cardiac function per se such as titrating HIF antagonism to ejection fraction or other more sophisticated markers of cardiac function (e.g. tissue Doppler, strain rate or speckle tracking echo).

Finally, it may also be pertinent to note HIF's dual roles in the heart (i.e. beneficial and detrimental). Accordingly, the use HIF antagonists may be better applied intermittently rather continuously in heart failure (Cai Z Circulation. (2003) 8; 108:79-85.)

The methods of the invention will now be illustrated by the following examples which are not intended to limiting. The scope of the invention is defined by the appended claims.

### Example 1

### HIF Upregulation can Contribute to Ventricular Dysfunction

As demonstrated in the diagram above, a central theme of this invention is that HIF upregulation directly contributes to the pathogenesis and hence exacerbation of ventricular dysfunction and heart failure. Although two models of putative HIF upregulation are indeed known to predispose to heart failure, they are both compromised by significant confounding effects.

The first, that of the ablation of PHD2 (one of the main enzymes that breaks down HIFs), does indeed cause heart failure. However the animals are afflicted by polycythaemia. This increase in red blood cell mass is known to be detrimental to the heart and the authors cite this as the most likely cause of heart failure (Minamishima YA Blood. (2008) 111:3236-44).

Similarly, a recent model of VHL knockout (VHL KO—VHL being another key degrader of HIF) also develops heart failure that can be specifically rescued by HIF-1α ablation. While this provides compelling evidence that specifically HIF-1α contributes to heart failure in this model, this model is highly confounded and compromised by the profound changes that occur in the VHL knockout including the development of cardiac de-differentiation (Lei L. Molecular Cell Biology. (2008)). The applicability of this model to general cardiac physiology is unclear.

In order to assess this question in a more physiological manner, we proposed that cardiac dysfunction would result from PHD3 knockout (KO). Since PHD3 is a less prominent HIF degrading enzyme in the heart (there is also substantial PHD2 in the heart), its ablation should provide a more graded, less confounded, increase in HIF signalling. Accordingly, as demonstrated in the diagram above, it was hypothesised that PHD3 KO's should develop heart failure, albeit in a gradual manner.

Making the PHD3 KO Animals

For construction of the PHD3 targeting vector, the following fragments were cloned in a pPNTLox2 vector (from 5′ to 3′): a 3.4 Kb BamH1 fragment located 3.1 Kb upstream of exon 1 (5′ flank), a neomycin resistance cassette in opposite orientation; a 3.9 Kb EcoR1 fragment located 1.4 Kb downstream of exon 1 (3′ flank), and a thymidine kinase selection cassette (Bishop T et al. Mol Cell Biol. 2008 Mar. 10). ES cells (129 SvEv background) were electroporated with the linearised targeting vector for PHD3 as described. Resistant clones were screened for homologous recombination by Southern blotting and PCR. Correctly recombined ES cells were then aggregated with morula stage embryos. To obtain PHD3+/−germline offspring in a 50% Swiss/50% 129 SvEv background, chimaeric male mice were intercrossed with wild-type Swiss female mice.

Assessing the PHD3 KO Animals

To assess the animals for ventricular dysfunction and heart failure, ˜11 month old wild-type and knockout animals were studied by 2D echocardiography and invasive hemodynamics. Mice were imaged under isoflurane anesthesia. A single echocardiographer blinded to mouse genotype performed all image acquisitions and analyses. After the echocardiographic study, a 1.4 F micromanometer conductance catheter (SPR-839, Millar Instruments Inc) was advanced from the right carotid artery into the LV and positioned under echocardiographic guidance. Measurements were recorded on a PowerLab 4SP data recorder (AD Instruments). LV systolic performance was assessed by dP/dtmax (mm Hg/s); LV diastolic performance dP/dtmin (mm Hg/s) and LV end-diastolic pressure (mm Hg).

PHD KO Causes Ventricular Dysfunction.

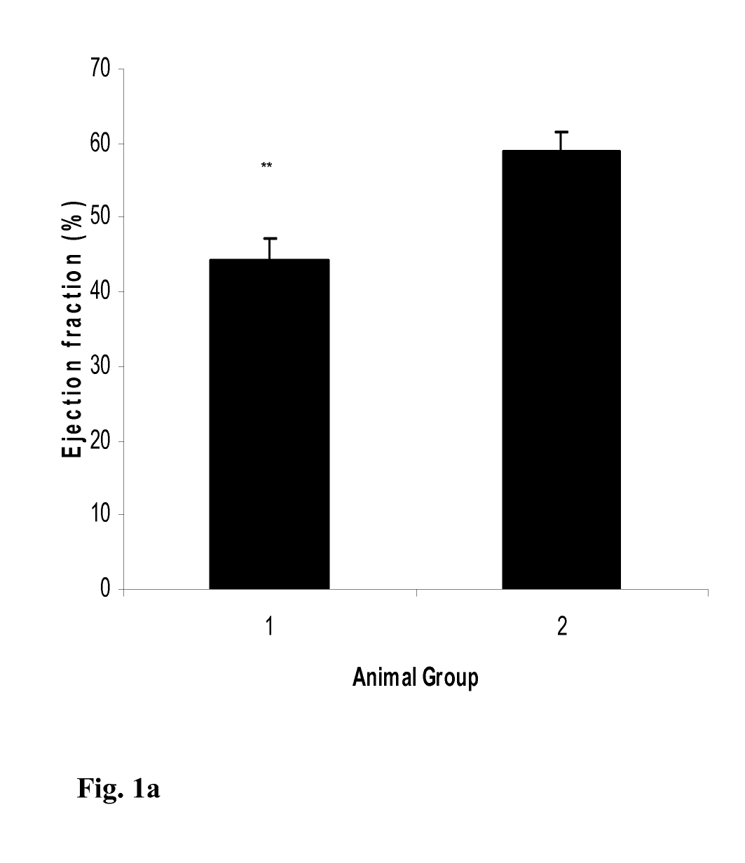

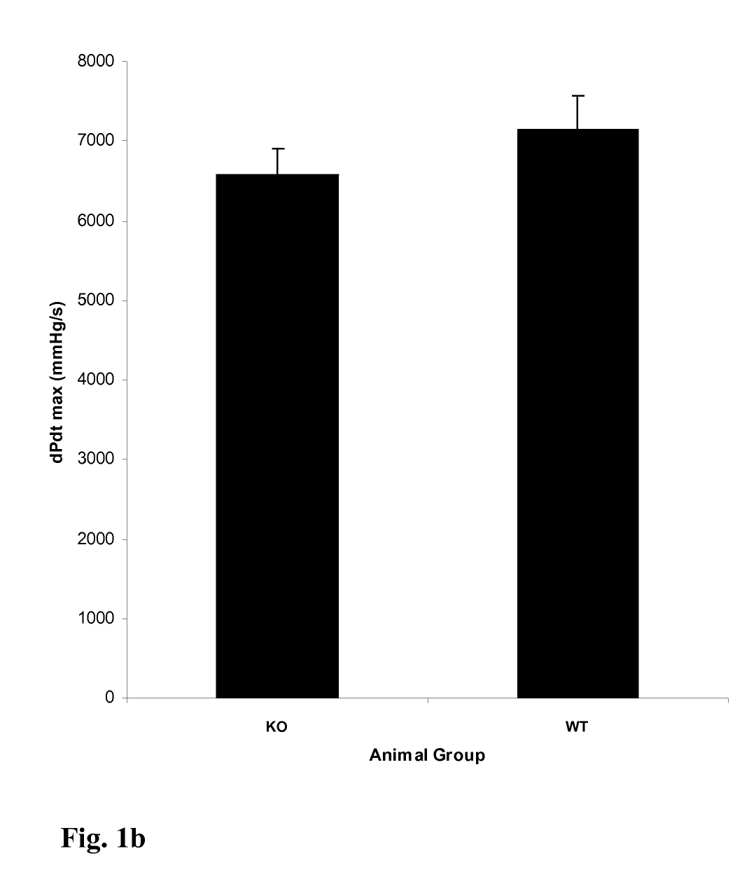

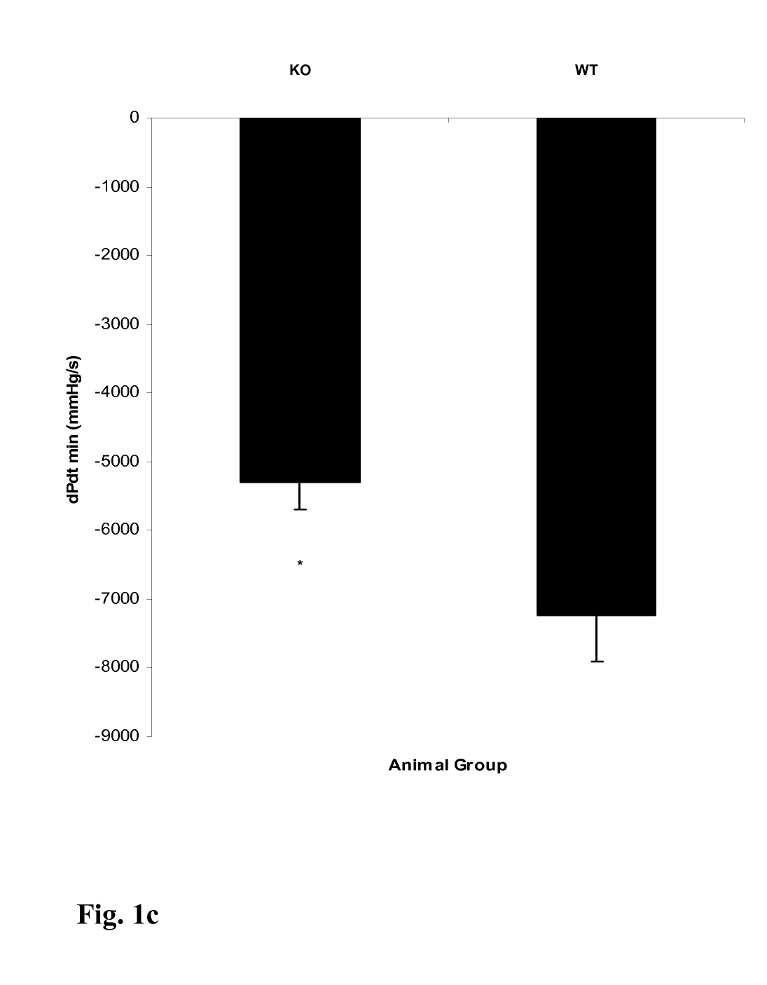

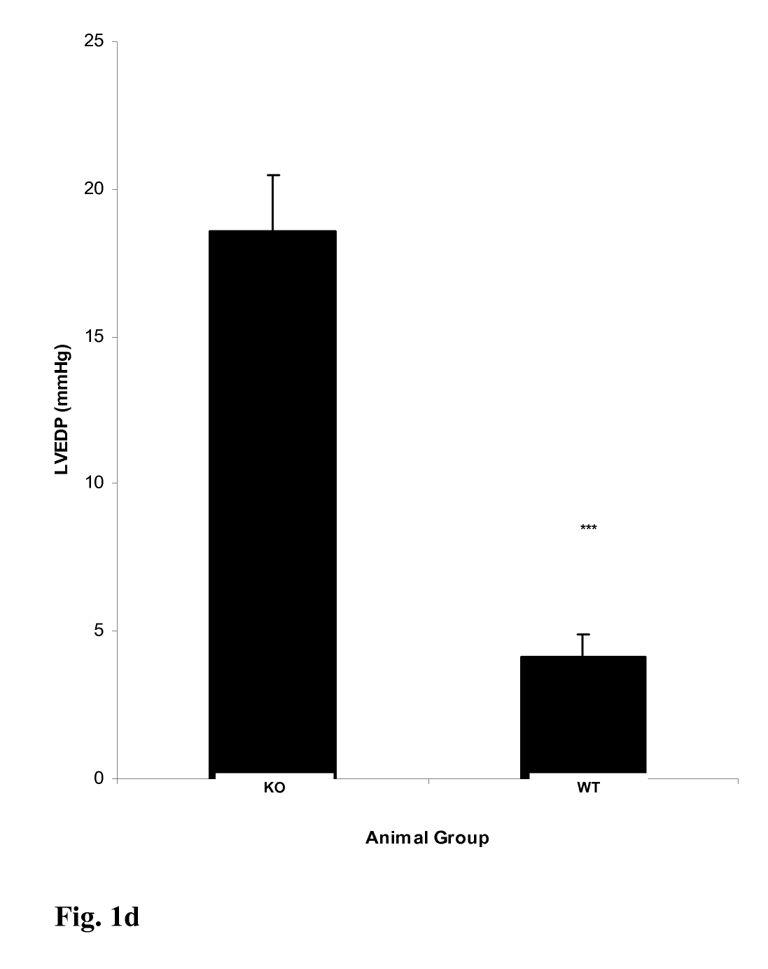

As can be seen from the Figures below (1a-d), it is clear that PHD3 KO causes at least moderate ventricular dysfunction. This clearly manifests by:
systolic dysfunction:
   FIG. 1a shows the ejection fraction (EF) as judged by 2D echocardiography and suggests that PHD3KO animals manifest moderate impairment of LV function (p<0.003) EF-WT 59%, EF-PHD3 KO 44% p<0.003, and
   FIG. 1b shows the assessment of LV systolic function by invasive hemodynamics and also suggests a trend towards a reduction of LV function, though this is not statistically significant (this discrepancy in EF and hemodyanmics may relate to loading and geometric conditions (radial vs longitudinal contraction)) (dpdt max (mmHg/s) WT: 7135, PHD3 KO: 6568);
diastolic dysfunction:
   FIG. 1c shows the assessment of LV diastolic function by invasive haemodynamics and suggests an impairment in LV relaxation and diastolic function (p<0.05) (dpdt min (mmHg/s) WT: −7241, PHD3 KO: −5298 p<0.05), and
   a raised LVEDP: FIG. 1d confirms impairment of LV dysfunction by a profound increase in LVEDP (p<0.0001) (WT 4 mmHg, PHD3 KO 19 mmHg p<0.0001).

The discrepancy between EF and dPdt max is well explained by the profound differences in preload (Van den Bergh A, Pflugers Arch. (2008) 455:987-94.); in this animal model there are no discernable differences in afterload (known to have a signficant effect on EF values) but a >4-fold difference in preload. Since dPdt max is preload dependant—the preserved dp/dt may reflect the profound increase in preload.

### Example 2

### The Effect of HIF-1α Overexpression on Cardiac Remodelling in HF

To assess the role of HIF-1α overexpression in HF, we will use mice homozygous for PHD2 flanked by loxp sites crossed with animals expressing α-MHC-cre driven by a modified oestrogen promoter (α-MHC[MER-Cre-MER]) to conditionally eliminate cardiac PHD\tilde{2}. Although these animals will overexpress other putative PHD2 targets in addition to HIFs, unlike the transgenic HIF-1α overexpressant (Kido, M. et al. J Am Coll Cardiol 46, 2116-2124 (2005)) this model is not limited by developmental chronic HIF overexpression, but expresses increased HIF in a temporally and tissue controlled manner. It is also less affected by post-translational HIF regulation. We already retain this cross on a C57/BL6 background. On being gavaged tamoxifen (20 mg/mL in corn oil, 1 mg/d for 6 days) cardiac specific PHD2 KO will be generated; albeit in a variegated manner (it is known that the efficiency of cre-induced recombination is not 100%) (Takeda, K. et al. Circulation 116, 774-781 (2007)). As detailed in the table below, untreated animals will undergo sham or MI-HF surgery (Dawson, D. et al. Circulation 112, 3729-3737 (2005)). Animals will be allowed to convalesce for 4 weeks, during which the affects of acute ischaemia will subside (Kido, M. et al. J Am Coll Cardiol 46, 2116-2124 (2005)). Animals will then be given tamoxifen and observed for 8 weeks. Cre expressing animals given tamoxifen, are used as controls in light of the reports suggesting potential for cre-cardiotoxcity (Xiong, S. et al. Circulation 115, 2925-2930 (2007), Nature 449, 378 (2007)).

| | Surgery | | | |
|---|---|---|---|---|
| | | | MI (Coronary artery | |
| | Sham Surgery | | ligation model) | |
| Background | WT | PHD2f/f | WT | PHD2f/f |
| | MerCreMer | MerCreMer | MerCreMer | MerCreMer |
| Treatment | Tamoxifen | Tamoxifen | Tamoxifen | Tamoxifen |

These animals will be assessed using the endpoints of echocardiography, invasive hemodynamics, MRI/MRS and histomorphometry. Although our surgical technique uses LAD landmarks to identify the ligation site in order to reduce infarct size variability (large infarcts are often fatal; small ones do not adverse remodel), the heterogeneity of resulting infarct size (between 10-70%, even when sutures are placed in identical locations due to coronary branching differences between even littermates) (Lygate, C. Am J Physiol Heart Circ Physiol 293, H3221 (2007)) has led our standard to be to assess infarct size by 3D echo within days after surgery and to retain animals with optimal infarct sizes (˜30%). The study will be powered for the least sensitive parameter of ejection fraction (EF) by 2D echo; to see a deterioration in EF of 5% (e.g. EF of 35% to 30%—with absolute sds of ˜6-5% on each of these values), for a study power of 80%, a sample size of 22 animals will be required in surgical groups (˜40% peri-surgical mortality) and 15 animals will be required in sham groups (assuming ˜15% peri-surgical mortality). Echocardiography (and MRI/MRS) will be performed under light anaesthetic using 1-1.5% isoflurane, and imaged using an Agilent Sonos 5500 with 6-15 MHz linear array transducer (Dawson, D. et al. Circulation 110, 1632-1637 (2004)). Hemodynamic indices will be measured using a 1.4 F Millar Mikro-tip catheter (SPR-671) via the carotid artery. The animal will ultimately be euthanized and after heart weight assessment, the hearts are frozen in liquid nitrogen or rinsed and fixed with 4% paraformaldehyde for histology.

### Example 3

### The Effect of HIF-1 α Ablation on Cardiac Remodelling in HF

To assess the cardiotoxic role of HIF-1α in acquired HF, we will ablate it using mice homozygous for exon 2 of HIF-1α flanked by loxp sites (Huang, Y. et al. FASEB J 18, 1138-1140 (2004)) crossed with animals expressing α-MHC[MER-Cre-MER] to conditionally eliminate cardiac HIF-1α.~ Importantly, these animals will not suffer ill from not being able to mobilize HIF-1α during development or during infarction, but will only have reduced HIF-1α during the adverse remodelling post-infarction. We already retain this cross on a C57/BL6 background. On being gavaged tamoxifen (20 mg/mL in corn oil, 1 mg/d for 6 days) cardiac specific HIF-1α KO will be generated; albeit again in a variegated manner (Takeda, K. et al. Circulation 116, 774-781 (2007)). As detailed in the table below, untreated animals will undergo sham or MI-HF surgery (Dawson, D. et al. Circulation 112, 3729-3737 (2005)). Animals will again be allowed to convalesce for 4 weeks, during which the affects of acute ischaemia will subside (Kido, M. et al. J Am Coll Cardiol 46, 2116-2124 (2005)). Animals will then be given tamoxifen and observed for 8 weeks.

| | Surgery | | | |
|---|---|---|---|---|
| | | | MI (Standard coronary artery | |
| | Sham Surgery | | ligation model) | |
| Background | WT | HIF-1α f/f | WT | HIF-1α f/f |
| | MerCreMer | MerCreMer | MerCreMer | MerCreMer |
| Treatment | Tamoxifen | Tamoxifen | Tamoxifen | Tamoxifen |

The details of the surgery and echocardiography are as above in Example 2. This part of the study will also be powered for EF by 2D echo; to see an improvement in EF of 5% (e.g. EF of 35% to 40%—with absolute standards errors of ˜6-7% on each of these values), for a study power of 80%, a similar sample size of 22 animals in surgical groups and 15 in each of the sham groups will be required in each group. Both studies will be assessed with statistically using the student's t-test and ANOVA where appropriate with post-test fishers analysis to assess significance at p<0.05 level.

### Example 4

### The Mechanisms of HIF-1α Cardiotoxicity in CHF

Although the above studies address the question of whether HIF augmentation in HF is detrimental to the remodelling pursuant to infarction they do not delineate the mechanisms of these adverse effects.

Validation: In order to validate the transgenic HIF manipulations, it will be essential to assess myocardial HIF levels and HIF regulated genes in both genetically modified and control animals. In order to do so, total RNA will be extracted from LVs using the Trizol based MiRNeasy Mini-Kits (Qiagen) yielding both micro and mRNA and involving treatment with DNase I. In recent pilot studies, despite the loss in yield due to the use of columns, we were able to extract 2-4 μg of total RNA from 10 mg of murine LV. These samples were bioanalysed (Agilent) and yielded RINS of >8; they were thus suitable and were successfully used for both mRNA/μRNA arrays and qtRT-PCR. (Qiagen Quantitect SYBR Green RTPCR kit) will be performed at a final volume of 20 μl with primers for HIF, VEGF and GLUT-1 (these primers cross intron-exon boundaries to reduce contaminant DNA amplification). Target gene results will be normalized to expression levels of the 36B4 RNA housekeeping gene and related to 100% in WT mice. Western Blot analysis will be performed to assess HIF-1α levels. Since HIF-1α is labile, LV powdered on dry ice will rapidly be exposed to 4M urea buffer containing protease inhibitor cocktail (Roche Diagnostics). 20 μgrams of total protein will be electrophoresed. We have found that overnight transfer is optimal, we prefer blocking in 5% nonfat milk in TBS-Tween for 1 h at room temperature. We incubate with anti HIF-1 primary antibody (Novus Biologicals) in TBS-Tween20 (5% mild) at 4° C. overnight. To further optimize the signal we use ECL advance (Amersham) after the secondary antibody.

Micro and mRNA Arrays: Although many of the acute hypoxic changes are mediated post-translationally, chronic changes in hypoxic signalling are likely to be manifest at the μ and mRNA level. Concordant with our own experience, we will send 1.2 μg RNA (30 μl of 40 ng/μl) for n=5 per group for both μ and mRNA analysis. Briefly, the total RNA will be labelled using TotalPrep RNA Amplification Kit (Ambion); the resulting biotin-labeled cRNA will be hybridized (16 h) to Illumina's Expression BeadChips. The hybridized cRNA will be detected by streptavidin-Cy3 and quantitated using BeadStation. Validation of the mRNA changes will be by q-RT-PCR as above and the μRNA by TaqMan miRNA qPCR.

Histological Analyses Fixed, paraffin embedded LV tissue slices will be studied by routine light microscopy stained by H&E, lipid-O red and trichrome to assess any changes in structure or lipid accumulation (Lei, L. et al. Mol Cell Biol (2008)). TUNEL assays will be performed using avidin biotin complex and diaminobenzidine kits from Vector Labs. LV capillary density will be determined by monoclonal PECAM-1 antibodies (Invitrogen) in WT and controls; digital images from 5 separate 40× fields will be assessed and counted.

MRI & MRS: Since HF is characterized by energy deficiency and chronic HIF activation putatively further compromises mitochondrial function, in vivo cardiac MRI/31P-S will be used to assess the models above. Although most previous MRS studies have required ex vivo analyses, recently developed technology has provided in vivo proton MR spectroscopy (1H-MRS) to investigate cardiac metabolism using an 11.7 T (500 MHz) MR with a single-voxel technique. Consistent with recent progress in other units, we have in vivo 31P-MRS for absolute quantification of metabolite concentrations (PCr, ATP, inorganic phosphate (Pi) and intracellular pH). Accordingly, MRI/31P-S will permit both further accurate assessment of cardiac function; PCr/ATP ratios and ΔG(ATP) hydrolysis at baseline and with inotropic stimulation to intensively assess myocardial energetics.

1. A method of treating heart failure or disorders associated with heart failure in a patient suffering from such a condition which comprises administering to such a patient a therapeutically effective amount of an agent that inhibits hypoxia-inducible factor (HIF).

2. A method according to claim 1 wherein the agent inhibits HIF-1.

3. A method according to claim 2 wherein the agent inhibits HIF-1α.

4. A method according to claim 1 wherein the agent inhibits the transcription of HIF, inhibits the translation of HIF, inhibits the subcellular localization of HIF, inhibits the activity of HIF, accelerates the degradation of HIF or decreases the stability of HIF.

5. A method according to claim 1 wherein the agent is a small molecule inhibitor of HIF.

6. A method according to claim 5 wherein the small molecule is taxol, rapamycin, 17-AAG (17-allylaminogeldanamycin), YC-1 (3-15′-hydroxymethyl-2′-furyl)-1-benzylindazole), adenosine, topotecan, imatinib mesylate, trastuzumab, NS398, celecoxib, ibuprofen, resteratiol, genistein, and berberin

7. The method according to claim 1 wherein the agent is a short hairpin RNA (shRNA) for a HIF gene.

8. The method according to claim 1 wherein the agent is a microRNA for a HIF gene.

9. The method according to claim 1 wherein the agent is an antisense oligonucleotide for a HIF gene.

10. The method according to claim 1 wherein the agent is a small double stranded interference RNA (siRNA) directed against a HIF gene.

11. The method according to claim 1 wherein agent is a monoclonal antibody directed against a HIF protein.

12. A method according to claim 1 wherein the heart failure is chronic heart failure.

13. A method according to claim 1 wherein the disorder associated with heart failure is diastolic heart failure or dilated, restrictive or hypertrophic cardiomyopathy.

14. An agent that inhibits hypoxia-inducible factor (HIF) for the treatment of heart failure or disorders associated with heart failure.
